(19) United States
(12) Patent Application Publication
(10) Pub. No.: US 2004/0254581 A1
(43) Pub. Date: Dec. 16, 2004

(54) Furcated bone screw

(76) Inventor: Walter J. Leclair, Shrewsbury, MA (US)

Correspondence Address: LAHIVE & COCKFIELD, LLP., 28 STATE STREET, BOSTON, MA, 02109, US

(21) Appl. No.: 10/766,136

(22) Filed: Jan. 27, 2004

Related U.S. Application Data
(60) Provisional application No. 60/444,953, filed on Feb. 4, 2003.

(51) Int. Cl. A61B 17/86

(52) U.S. Cl. 606/73

(57) ABSTRACT

A furcated orthopedic bone screw includes a threaded portion that is cut or slotted into multiple radial segments. The furcated bone screw can be bifurcated, trifurcated, or have a number of radial segments greater than two or three. Each of the radial segments of the furcated bone screw is plastically deformed in a radially outward direction to create a compressible screw tip of increased diameter over the non-furcated portion of the bone screw. The resilient and elastic properties of the furcated bone screw material enable the compression of the radial segments during insertion of the furcated bone screw into a bone or prosthetic. Once inserted, the resilient properties of the furcated bone screw material cause the radial segments of the screw tip to return to their plastically deformed radially expanded state, thus causing a radial force against the screw hole and the bone or prosthetic. The radial force of the threaded radial segments results in the furcated bone screw being better able to engage the bone or prosthetic to resist pull-out and compensate for stripped threads or soft bone.

### RELATED APPLICATION

This application claims priority to and the benefit of co-pending U.S. Provisional application No. 60/444,953 filed Feb. 4, 2003, which is expressly and entirely incorporated herein by reference.

### FIELD OF THE INVENTION

The present invention relates to orthopedic prosthetics, and more particularly to a furcated screw for fixing an orthopedic prosthesis to a bone.

### BACKGROUND OF THE INVENTION

Stainless steel, titanium, and titanium alloy bone screws are conventional in the practice of orthopedic surgery. The bone screws secure orthopedic plates, pins, and rods to bone, in addition to repairing fractured bone directly. Osteoporosis, osteomalacia, disuse osteopenia, and other known diseases of bone can cause the bone to become less dense, thinner, or softer than normal healthy bone. When existing screws are placed into osteoporotic or soft bone, the screws tend to strip their threads and/or pull out of the bone. This is a function of the inferior strength of the soft bone, and occurs with screws of different diameter, metal composition, and thread pitch. The result is an inferior screw fixation in the bone. In most instances, screws having a larger diameter thread cannot replace the stripped screws because additional constraints, such as the diameter of the plate or orthopedic implant holes through which the screw must pass or the size of the bone fragments, prohibit the use of a larger screw.

Conventional surgical solutions to this problem include repositioning plates, rods, and/or drill holes to allow repositioning or redirection of the screws, placement of a nut on a protruding screw tip, or cementing of the screw in place with, e.g., polymethylmethacralate bone cement. Such conventional approaches can create additional damage to the circulation through the bone and other tissues, or increase the risk of damage to neurovascular structures with the additional required surgery. Bone cement fixation introduces additional foreign bodies into the bone and can cause thermal damage to the living bone during the exothermic reaction that occurs during the cement curing process. When presented with the situation of stripped threads in a fixed diameter orothopedic bone screw, a solution is required that does not necessitate significant additional surgical time, cause additional tissue damage, or cause neurovascular physiologic trauma.

### SUMMARY OF THE INVENTION

It is therefore desirable to have a screw capable of passing through a hole having a first diameter, and expanding to a larger screw diameter after passing through the initial opening of the hole, once there is a reduced compressive force acting on the screw. The present invention provides a solution to address this need.

In accordance with one embodiment of the present invention, a furcated bone screw includes a shaft having a first end and a second end. A screw thread circumnavigates the shaft. A plurality of elongate slots are longitudinally formed in the shaft from the second end and create a plurality of furcated branches. The plurality of branches are plastically deformed radially outwardly from a first screw diameter to a second circumferential diameter and are compressible to the first screw diameter without plastic deformation.

In accordance with aspects of the present invention, the plurality of furcated branches can compress to the first screw diameter state when furcated bone screw is initially positioned at the opening of a hole. The plurality of furcated branches return to the second circumferential diameter upon reduction of a radially compressive force. The plurality of furcated branches can extend for a distance of at least half of the length of the shaft.

In accordance with further aspects of the present invention, the screw thread extends from the second end of the shaft at least substantially to the first end of the shaft. A screw head can be disposed at the first end of the shaft. A driver can be disposed at the first end of the shaft.

In accordance with further aspects of the present invention, the plurality of furcated branches is formed of three branches. The plurality of furcated branches have sufficient flexibility such that the plurality of branches are compressible by a user. The furcated bone screw can be formed at least partially by titanium.

In accordance with one embodiment of the present invention, a furcated bone screw includes a shaft having a first end and a second end. A screw thread circumnavigates the shaft. A plurality of elongate slots is longitudinally formed in the shaft from the second end creating a furcated means. The furcated means extend radially outwardly and are compressible.

In accordance with aspects of the present invention, the furcated means can compress from a circumferential diameter at the radially outwardly configuration to a relatively smaller screw diameter when the furcated bone screw is initially positioned at the opening of a hole. The furcated means can return to a radially expanded state upon reduction of a radially compressive force acting on the furcated means.

### BRIEF DESCRIPTION OF THE DRAWINGS

The present invention will become better understood with reference to the following description and accompanying drawings, wherein:

FIG. 1 is a cross-sectional side view of the furcated bone screw, according to one aspect of the present invention;

FIG. 2 is a perspective top view of the furcated bone screw, according to one aspect of the present invention;

FIG. 3 is a perspective illustration of the furcated bone screw in compressed state, according to one aspect of the present invention;

FIG. 4 is a perspective illustration of the furcated bone screw in expanded state, according to one aspect of the present invention; and

FIG. 5 is a perspective illustration of two furcated bone screws inserted into a bone, according to one aspect of the present invention.

### DETAILED DESCRIPTION

An illustrative embodiment of the present invention relates to a furcated or branched orthopedic bone screw. The furcated bone screw includes a threaded portion that is cut or slotted into multiple radial segments. The furcated bone screw can be bifurcated, trifurcated, or have a number of radial segments greater than two or three. Each of the radial segments of the furcated bone screw is plastically deformed in a radially outward direction to create a compressible screw tip of increased diameter over the non-furcated portion of the bone screw. The resilient and elastic properties of the furcated bone screw material enable the compression of the radial segments during insertion of the furcated bone screw into a bone or prosthetic. Once inserted, the resilient properties of the furcated bone screw material cause the radial segments of the screw tip to return to their plastically deformed radially expanded state, thus causing a radial force against the screw hole and the bone or prosthetic. The radial force of the threaded radial segments results in the furcated bone screw being better able to engage the bone or prosthetic to resist pull-out and compensate for stripped threads.

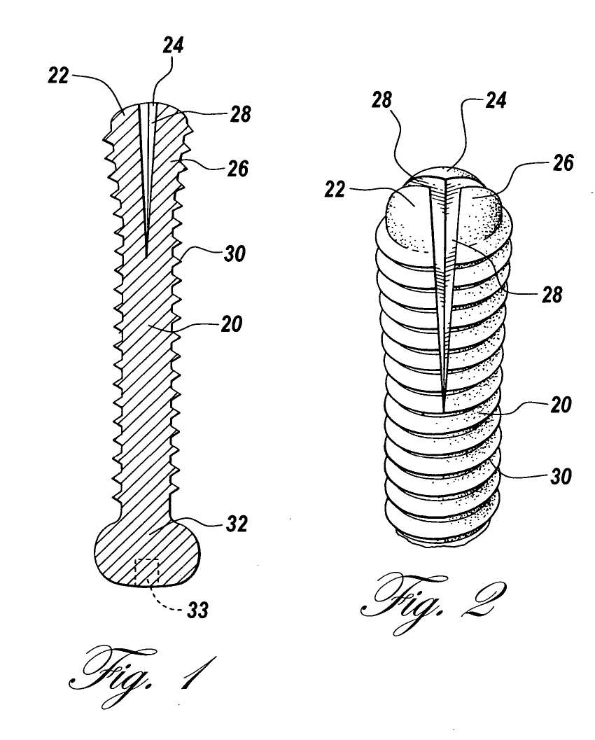

FIGS. 1 through 5, wherein like parts are designated by like reference numerals throughout, illustrate an example embodiment of a furcated bone screw, according to the present invention. Although the present invention will be described with reference to the example embodiment illustrated in the figures, it should be understood that many alternative forms can embody the present invention. One of ordinary skill in the art will additionally appreciate different ways to alter the parameters of the embodiments disclosed, such as the size, shape, or type of elements or materials, in a manner still in keeping with the spirit and scope of the present invention.

FIGS. 1 and 2 illustrate a furcated bone screw 20 in accordance with the present invention. As illustrated, the furcated bone screw 20 has a first branch 22, a second branch 24, and a third branch 26. However, one of ordinary skill in the art will appreciate that there can be any number of branches, ranging from two, to three, to greater than three. The first, second, and third branches 22, 24, and 26 are formed by the creation of multiple elongate slots 28. The elongate slots 28 extend through a portion of the furcated bone screw 20, but do not extend completely from one end to the other of the furcated bone screw 20.

The furcated bone screw 20 further includes a series of circumnavigating threads 30. The threads 30 can extend at least substantially along the length of the bone screw 20. More specifically, the threads 30 can extend for a substantial portion of the length of the furcated bone screw 20, either completely from one end to the other, or leaving a small section unthreaded (such as with a common lag bolt). As understood by one of ordinary skill in the art, the threads 30 provide the fixing or gripping action of the furcated bone screw 20 as it is inserted into bone or implant. As with other screws, the furcated bone screw 20 includes a screw head 32 with a driver bore 33 for receiving a tool for tightening or loosing the furcated bone screw 20.

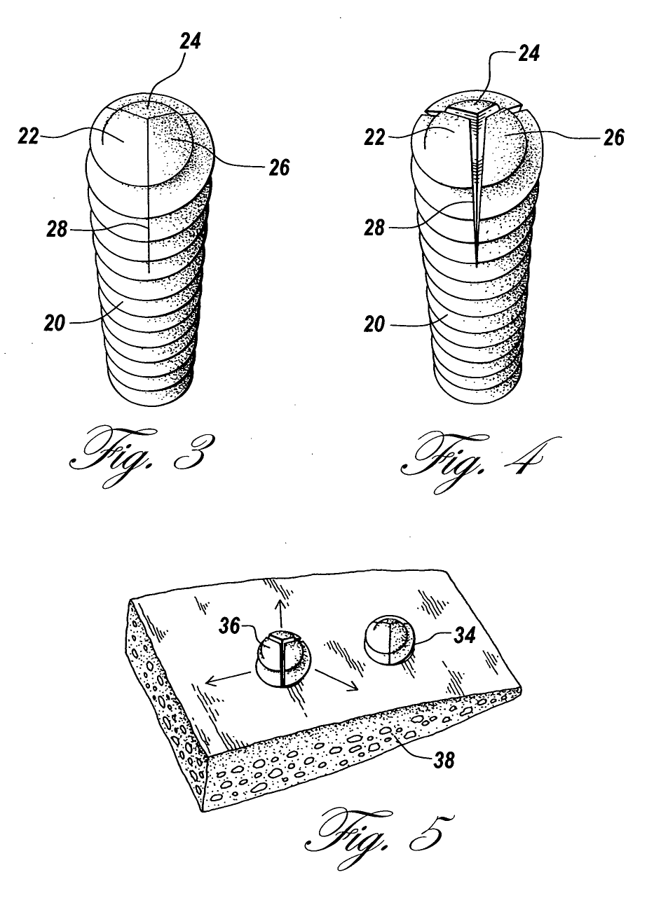

During manufacture, a screw is first formed without the elongate slots 28. The elongate slots 28 are then added, such as by using a cutting tool as known to one of ordinary skill in the art, forming the desired number of branches for the furcated bone screw 20. For example, the first branch 22, the second branch 24, and the third branch 26 can be formed in accordance with the example illustrated embodiment as shown in FIG. 3. Each of the branches 22, 24, and 26 is then plastically deformed radially outwardly. As such, in a steady state, the first branch 22, second branch 24, and third branch 26 each bow radially outwardly as depicted in FIG. 4. The furcated bone screw 20 is then ready for use in a bone or orthopedic implant. It should be noted that the branches 22, 24, and 26 are plastically deformed radially outwardly only to the extent that when the branches 22, 24, and 26 are compressed back to their original position, such compression does not cause additional plastic deformation. In other words, after the branches 22, 24, and 26 have been plastically deformed outwardly, if an external force is applied that returns the branches 22, 24, and 26 back to their original position (such as if the furcated screw 20 is forced into a hole of a diameter sized for the screw) once that external force is removed, the branches 22, 24, and 26 return to their plastically deformed outward position.

FIG. 5 illustrates a first furcated bone screw 34 and a second furcated bone screw 36. The first furcated bone screw 34 is shown just beginning to pass through a bone 38. The external radial forces on the first furcated bone screw 34 in this position have maintained the first furcated bone screw 34 in a compressed state. Contrarily, the second furcated bone screw 36 has progressed further through the bone 38 and has begun to expand each of its branches in the direction of the arrows shown, back to the plastically deformed position. This expansion action can take place within the hole formed in the bone 38 as well. The expansion creates a larger diameter screw, which better resists pull-out from the bone and can compensate for stripped threads.

In the instance of the furcated bone screw 20 being screwed into a hole having stripped threads, the furcated bone screw 20 can expand each of its branches as the stripped hole allows. In other words, the softer bone, or a hole with stripped threads, can not resist the expansion forces of the branches 22, 24, and 26. As such, once a user begins driving the furcated bone screw 20 into the bone, the branches 22, 24, and 26 begin their expansion. The expansion action fills up any larger diameter within the hole, or fills up the space caused by the stripped threads, and the furcated bone screw 20 fixes itself to the bone.

Accordingly, the furcated bone screw 20 has the ability to fit through a hole, such as a hole in a plate, and then after passing through the initial opening of the hole, the branches 22, 23, and 26 of the furcated bone screw 20 can expand outwardly to fill any void or soft bone space having a larger diameter than that of the hole.

The furcated bone screw 20 can be manufactured using a number of different methods and materials as understood by one of ordinary skill in the art. Suitable materials have sufficient resiliency to return to the expanded steady state after compression, and also have sufficient elasticity to be plastically deformed during manufacture. Some example materials can include, but are not limited to, stainless steel, titanium, and titanium alloys. The specific material of the furcated bone screw 20 can be chosen based on a desire to match the screw material to the material of an existing plate or other implant, to avoid electrolyte defects, as is understood by one of ordinary skill in the art.

The threads on the furcated bone screw 20 can have different angles, pitch, depth, and other dimensions, such dimensions configuring the furcated bone screw 20 for different bone applications as would be understood by one of ordinary skill in the art. Furthermore, the branches of the furcated bone screw 20 can be formed by a number of different cutting devices, including but not limited to saws, blades, laser, and the like, as is understood by one of ordinary skill in the art. In addition, the furcated bone screw 20 can have a tapered diameter to improve the ability of the furcated bone screw 20 to expand within a hole, or hold its place within a hole after implantation.

The furcated bone screw of the present invention maintains a resiliency after plastic deformation that provides an outward radial force on each of the branches when compressed by a screw hole. The furcated bone screw is able to replace stripped bone screws without repositioning of the screw in the bone. Compression of the furcated bone screw tip allows insertion into plates, orthopedic implants, or existing screw holes in bones. Diameter taper improves resistance to pull-out. The configuration of the furcated bone screw can be used in screws of different materials, thus enabling one to match the screw material to an existing plate or other implant, avoiding electrolyte defects.

Numerous modifications and alternative embodiments of the present invention will be apparent to those skilled in the art in view of the foregoing description. Accordingly, this description is to be construed as illustrative only and is for the purpose of teaching those skilled in the art the best mode for carrying out the present invention. Details of the structure may vary substantially without departing from the spirit of the invention, and exclusive use of all modifications that come within the scope of the disclosed invention is reserved.

What is claimed is:

1. A furcated bone screw, comprising:
a shaft having a first end and a second end;
a screw thread circumnavigating the shaft; and
a plurality of elongate slots longitudinally formed in the shaft from the second end and creating a plurality of furcated branches;
wherein the plurality of branches are plastically deformed radially outwardly from a first screw diameter to a relatively larger second circumferential diameter and are compressible to the first screw diameter without plastic deformation.

2. The furcated bone screw of claim 1, wherein the plurality of furcated branches can compress to the first screw diameter state when furcated bone screw is initially positioned at the opening of a hole.

3. The furcated bone screw of claim 1, wherein the plurality of furcated branches return to the second circumferential diameter upon reduction of a radially compressive force.

4. The furcated bone screw of claim 1, wherein the plurality of furcated branches extend for a distance of at least half of the length of the shaft.

5. The furcated bone screw of claim 1, wherein the screw thread extends from the second end of the shaft at least substantially to the first end of the shaft.

6. The furcated bone screw of claim 1, further comprising a screw head disposed at the first end of the shaft.

7. The furcated bone screw of claim 1, further comprising a driver disposed at the first end of the shaft.

8. The furcated bone screw of claim 1, wherein the plurality of furcated branches comprises three branches.

9. The furcated bone screw of claim 1, wherein the plurality of furcated branches have sufficient flexibility such that the plurality of branches are compressible by a user.

10. The furcated bone screw of claim 1, wherein the furcated bone screw is formed at least partially by titanium.

11. A furcated bone screw, comprising:
a shaft having a first end and a second end;
a screw thread circumnavigating the shaft; and
a plurality of elongate slots longitudinally formed in the shaft from the second end and creating a furcated means;
wherein the furcated means extend radially outwardly and are compressible.

12. The furcated bone screw of claim 11, wherein the furcated means can compress from a circumferential diameter at the radially outwardly configuration to a relatively smaller screw diameter when the furcated bone screw is initially positioned at the opening of a hole.

13. The furcated bone screw of claim 11, wherein the furcated means can return to a radially expanded state upon reduction of a radially compressive force acting on the furcated means.

14. The furcated bone screw of claim 11, wherein the furcated means are formed along at least half of the length of the shaft.

15. The furcated bone screw of claim 11, wherein the screw thread extends from the second end of the shaft at least substantially to the first end of the shaft.

16. The furcated bone screw of claim 11, further comprising a screw head disposed at the first end of the shaft.

17. The furcated bone screw of claim 11, further comprising a driver disposed at the first end of the shaft.

18. The furcated bone screw of claim 11, wherein the furcated means comprises a plurality of furcated branches.

19. The furcated bone screw of claim 11, wherein the furcated means have flexibility such that the furcated means are compressible by a user.

20. The furcated bone screw of claim 11, wherein the furcated bone screw is formed at least partially by titanium.
